(19) United States
(12) Patent Application Publication
(10) Pub. No.: US 2008/0004665 A1
(43) Pub. Date: Jan. 3, 2008

(54) Determination of cardiac pacing parameters based on non-localized sensing

(76) Inventors: Aaron R. McCabe, Minneapolis, MN (US); Alok S. Sathaye, Minneapolis, MN (US); Yinghong Yu, Shoreview, MN (US)

Correspondence Address: HOLLINGSWORTH & FUNK, LLC, Suite 125, 8009 34th Avenue South, Minneapolis, MN, 55425, US

(21) Appl. No.: 11/478,428

(22) Filed: Jun. 29, 2006

(51) Int. Cl. A61N 1/00

(52) U.S. Cl. 607/9

(57) ABSTRACT

An atrioventricular delay (AVD) for cardiac pacing therapy is determined based, at least in part, on one or more cardiac activation signals sensed using body-implantable electrodes providing non-local sensing. A conduction delay is estimated using a non-local cardiac activation signal and the AVD is determined based on the conduction delay. Estimating the conduction delay may involve measuring a P-wave width or measuring a QRS complex width of the non-local signal. If multiple signals are sensed, the conduction delay may be estimated from a selected signal or may be estimated by forming a representative signal such as through averaging or other methods. The AVD may be determined as a function of the estimated conduction delay.

### FIELD OF THE INVENTION

The present invention relates generally to cardiac pacing therapy, and more specifically, to methods and systems for determination of pacing parameters.

### BACKGROUND OF THE INVENTION

Congestive heart failure is the loss of pumping power of the heart, resulting in the inability to deliver enough blood to meet the demands of peripheral tissues. Congestive heart failure (CHF) may cause weakness, loss of breath, and build up of fluids in the lungs and other body tissues.

CHF is usually a chronic, long term condition, but can occur suddenly. It may affect the left heart, right heart or both sides of the heart. Deterioration of the muscles of the heart result in an enlargement of the heart and reduced contractility. The reduced contractility decreases the cardiac output of blood and may result in an increased heart rate. Cardiac conduction path block may also occur in enlarged heart tissue, causing the signals that control the heart rhythm to travel more slowly through the enlarged heart tissue. For example, if CHF affects the left ventricle, signals that control the left ventricular contraction are delayed, and the left and right ventricles do not contract simultaneously. Non-simultaneous contractions of the left and right ventricles decrease the pumping efficiency of the heart.

CHF may be treated by medication and/or by cardiac pacing therapy. Pacing therapy to promote synchronization of heart chamber contractions for improved cardiac function is generally referred to as cardiac resynchronization therapy (CRT). Some cardiac pacemakers are capable of delivering CRT by pacing multiple heart chambers. Pacing pulses are delivered to the heart chambers in a sequence that causes the heart chambers to contract in synchrony, increasing the pumping power of the heart and delivering more blood to the peripheral tissues of the body. In the case of dysynchrony of right and left ventricular contractions, a biventricular pacing therapy may be used to resynchronize the left and right ventricles. Bi-atrial pacing or pacing of all four heart chambers may alternatively be used.

Pacing therapy is delivered by pacing one or more heart chambers using pacing delay intervals to control the timing and sequence of the pacing pulses. Appropriate specification of these pacing delay intervals is desirable to achieve optimal improvement of cardiac function through enhanced synchrony. For the reasons stated above, and for other reasons stated below which will become apparent to those skilled in the art upon reading the present specification, there is a need in the art for methods and systems that provide for determination of delay intervals for pacing. The present invention fulfills these and other needs.

### SUMMARY OF THE INVENTION

The present invention is directed to systems and methods for determining cardiac pacing parameters based at least in part on a cardiac signal sensed via an electrode pair configured to provide non-local sensing.

One embodiment of the invention involves a method for determining an atrioventricular delay (AVD) for cardiac pacing therapy. At least one cardiac activation signal is sensed using a body-implantable electrode pair providing non-local sensing. A conduction delay is estimated using the at least one non-local cardiac activation signal. The AVD is determined based on the conduction delay. According to various aspects, estimating the conduction delay may involve measuring a P-wave width or measuring a QRS complex width. According to some implementations, both the P-wave width and the QRS complex width are estimated using the non-local cardiac activation signal and are used to determine the AVD.

In one implementation, a plurality of cardiac activation signals are respectively sensed via a plurality of electrode pairs providing non-local sensing. Estimation of the conduction delay involves selecting a cardiac activation signal from the plurality of cardiac activation signals and measuring a feature of the selected cardiac activation signal. In one implementation, at least two cardiac activation signals are selected and the selected signals are combined to form a representative non-local cardiac activation signal. The conduction delay is estimated using the representative cardiac activation signal, such as by measuring a signal feature of the representative cardiac activation signal.

According to various aspects, the AVD may be determined as a function of the conduction delay. The function may involve a linear combination of factors including the conduction delay.

In one implementation, an atrial rate associated with the AVD may be determined. The AVD may be stored in memory indexed by the atrial rate. A pacing therapy, such as cardiac resynchronization therapy, may be delivered to the patient by selecting an AVD corresponding to the present atrial rate.

In some implementations, determination of the AVD is performed at least in part by a human analyst. In other implementation, determination of the AVD is performed at least in part by a processor.

In another aspect of the invention, information from non-local sensing may be combined with information from local sensing. According to this implementation, at least on cardiac activation signal is sensed using a cardiac electrode pair providing local sensing. an additional conduction parameter is estimated based on the local signal. The AVD is determined based on the conduction delay and the additional conduction parameter.

Another embodiment of the invention is directed to a cardiac rhythm management system. The system includes at least one pair of implantable electrodes configured to electrically couple to a heart and to provide non-local sensing of cardiac electrical activity. Sense circuitry is configured to sense at least one cardiac activation signal from the at least one electrode pair. A processor is configured to estimate a conduction delay based on the at least one cardiac activation signal and determines an AVD for cardiac pacing therapy based on the conduction delay. In various configurations, at least one electrode of the electrode pair may include an intracardiac electrode and/or may include a subcutaneous, non-intrathoracic electrode. According to one approach, the pacing therapy comprises biventricular pacing therapy.

The processor may be configured to determine the conduction delay based on a measured feature of the at least one cardiac activation signal, such as a measured P-wave width or a measured QRS complex width.

The processor may determine a plurality of AVDs, each AVD associated with an atrial rate. The system may include a memory configured to store the AVDs indexed by atrial rate.

In some configurations, the processor is at least in part patient-external. In other configurations, the processor is implantable within the body.

According to another implementation, the system may include one or more additional electrodes configured to electrically couple to the heart and to provide local sensing of cardiac electrical activity. The sense circuitry may be configured to sense at least one local cardiac activation signal via the one or more additional electrodes. The processor is configured to determine an additional conduction parameter based on the local cardiac activation signal and to determine the AVD based on the conduction delay and the additional conduction parameter.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1A is a flow diagram illustrating a method for determining the atrioventricular delay (AVD) using a cardiac signal sensed via a non-local electrode pair in accordance with embodiments of the invention;

FIG. 1B is a flow diagram illustrating a method for determining the AVD using a cardiac signal sensed via a non-local electrode pair and a cardiac signal sensed via a local electrode pair in accordance with embodiments of the invention;

FIGS. 2A and 2B are diagrams of a cardiac activation signal for three consecutive heartbeats (FIG. 2A) and a magnified portion of the signal for the first two consecutive beats (FIG. 2B);

FIG. 3 is a illustration of an implantable cardiac device including a lead assembly shown implanted in a sectional view of the heart, in accordance with embodiments of the invention;

FIG. 4 is a block diagram illustrating various components of a system for determining the AVD in accordance with embodiments of the invention;

FIGS. 5A-5D are flow diagrams illustrating methods for determining the AVD using at least a cardiac activation signal sensed via a non-local electrode pair in accordance with embodiments of the invention;

FIG. 6 is a polar plot of a cardiac vector superimposed over a frontal view of a thorax, with the origin of the polar plot located at the atrioventricular (AV) node of a patient's heart;

FIG. 7 is a polar plot of cardiac vectors obtained using a source separation in accordance with the present invention; and

FIG. 8 is a flow diagram of a method by which a device may implement selection of AVD based on atrial rate in accordance with embodiments of the invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof, and in which is shown by way of illustration, various embodiments in which the invention may be practiced. It is to be understood that other embodiments may be utilized, and structural and functional changes may be made without departing from the scope of the present invention.

Systems, devices or methods according to the present invention may include one or more of the features, structures, methods, or combinations thereof described hereinbelow. For example, a device or system may be implemented to include one or more of the advantageous features and/or processes described below. It is intended that such device or system need not include all of the features described herein, but may be implemented to include selected features that provide for useful structures and/or functionality. Such a device or system may be implemented to provide a variety of therapeutic or diagnostic functions.

Embodiments of the invention are directed to systems and methods for determining the atrioventricular pacing delay (AVD) to enhance cardiac function. For example, some implementations are directed to determination of an AVD to enhance delivery of cardiac resynchronization pacing therapy (CRT). CRT involves pacing stimulation applied to one or more heart chambers in a manner that compensates for conduction delays. CRT may involve pacing one or both atria and/or one or both ventricles.

CRT results in a more coordinated contraction of the ventricles with improved pumping efficiency and increased cardiac output. CRT can be achieved in certain patients by pacing at a single unconventional site, such as the left ventricle instead of the right ventricle. For example, the LV pace may be delivered after an appropriate delay initiated relative to a right ventricular (RV) sense or may be delivered after an appropriate delay initiated relative to an atrial sense or pace. In some configurations, resynchronization pacing may involve biventricular pacing with the paces to right and left ventricles delivered either simultaneously or sequentially, with the interventricular delay interval between the paces termed the biventricular offset (BVO).

In one example of CRT, right atrial paces and senses trigger an atrioventricular delay (AVD) which upon expiration results in a pace to one of the ventricles and which is stopped by a right ventricular sense. A pace to the contralateral ventricular is delivered at the specified BVO with respect to expiration of the AVD. As the term is used herein for biventricular pacing, the AVD refers to the interval between an atrial event (i.e., a pace or sense in one of the atria, usually the right atrium) and the first ventricular pace to one of the ventricles. The duration of the AVD may be the same or different depending upon whether it is initiated by an atrial sense or pace (i.e., in atrial tracking and AV sequential pacing modes, respectively).

Methods, devices, and systems of the present invention provide for determination of the AVD implemented in pacing therapy, such as the AVD used for cardiac resynchronization therapy. In accordance with embodiments of the invention, the AVD is determined using a cardiac electrical activation signal obtained via implantable electrodes that provide non-local sensing. In some embodiments, the AVD is determined using a cardiac electrical activation signal obtained via non-local sensing in conjunction with a cardiac electrical activation signal obtained via local sensing.

Cardiac electrical activation signals originate from electrophysiological signals originating in and propagated through the myocardium, which provide for the cardiac muscle contraction. Local sensing of cardiac activation signals may be achieved via electrodes that make contact with the cardiac myocardium. Non-local sensing of the cardiac activation signals may be achieved via electrodes that are coupled to, but do not make directed contact with, the cardiac myocardium. A sensed non-local cardiac activation signal is effectively a superposition of all the depolarizations occurring within the heart that are associated with cardiac contraction, along with noise components.

In implantable pacemakers, tip electrodes which are configured to make direct contact with the myocardium have traditionally been used for local sensing. Non-local sensing may be accomplished via various electrode pairs of an implantable pacemaker or defibrillator, such as RV-ring to LV-ring, RV-ring to RV-coil, RV-ring to SVC-coil, LV-ring to RV-coil, LV-ring to SVC-coil RV-ring to RA-ring, LV-ring to RA-ring, or may include sensing between two can electrodes, between a can electrode and an indifferent electrode, or between a can or indifferent electrode and a ring or coil electrode, for example. An electrode pair, as used herein, refers to at least two electrodes, but each electrode of the pair may comprise multiple electrodes or electrode elements used for sensing.

In some situations, the particular placement of a ring, coil, or other electrode configured for non-local sensing may cause the electrode to make direct contact with the myocardium. In these situations, unexpected local sensing is apparent from the cardiac waveform morphology and use of the electrode in conjunction with pacing parameter determination in accordance with embodiments of the present invention may be avoided.

Embodiments of the invention are directed to methods and systems for determining the AVD based at least in part on estimation of the heart's interchamber conduction delays. In some embodiments, the estimated conduction delays used for AVD determination involve measurements taken from cardiac activation signals obtained through non-local sensing. In some embodiments, information from cardiac signals obtained via local sensing is used in conjunction with the information from non-local cardiac activation signals. In one implementation, estimation of the RA-LA conduction timing involves measuring the P-wave width of a cardiac signal sensed via a non-local electrode pair. In one implementation, estimation of the RV-LV conduction timing involves measuring the QRS complex width of a non-local cardiac signal.

In some embodiments, sensing via multiple non-local pairs may be used to provide multiple non-local cardiac signals. According to various aspects, the P-wave width (Pwidth) used in the determination of the AVD may be based on the average P-wave width of two or more of the sensed signals; may be based on the longest P-wave width; may be based on the P-wave width of the least noisy signal; may be based on an average or other calculation involving two or more of the longest P-wave widths; may be based on an average or other calculation involving the P-wave widths of two or more least noisy signals; or may be based on other calculations using information from the non-local signal. The QRS width value may be determined from multiple non-local signals using similar approaches.

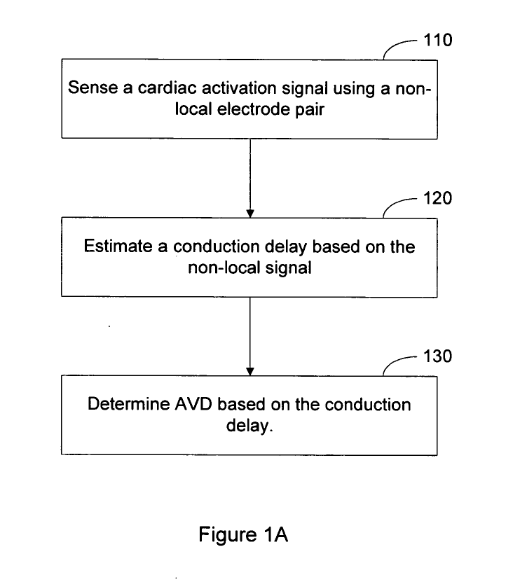

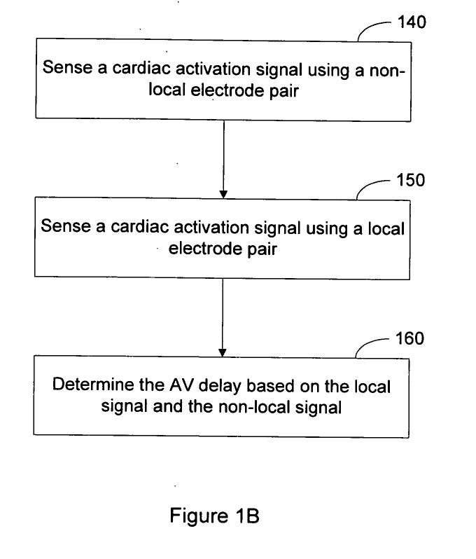

FIG. 1A is a flow diagram illustrating a method for determining AVD in accordance with embodiments of the invention. Implantable electrodes forming at least one non-local electrode pair is used for sensing 110 at least one cardiac activation signal. A conduction delay is estimated 120 based on the sensed cardiac activation signal. The estimated conduction delay may be an estimate of the conduction delay between a right atrial (RA) depolarization and a left atrial (LA) depolarization, a right atrial depolarization and a right ventricular (RV) polarization, a right atrial depolarization and a left ventricular (LV) depolarization, or right ventricular depolarization and a left ventricular depolarization, for example.

The AVD is determined 130, at least in part, using the conduction delay. The AVD so determined may be used in a pacing therapy to modify the electrical activation sequence of the cardiac rhythm to enhance cardiac pumping function. Modification of the electrical activation sequence changes the mechanical contractile sequence of the heart and, if effective, improves the patient's hemodynamic status.

In accordance with some embodiments, the AVD is determined based on both a non-local cardiac activation signal and a local signal. As previously discussed, local cardiac activation signals are sensed via an electrode pair that includes at least one electrode, such as a tip electrode, configured to make direct contact with the cardiac myocardium and thus provide local sensing. In accordance with the method illustrated in the flowchart of FIG. 1B, a cardiac activation signal is sensed via both a non-local electrode pair and a local electrode pair 140, 150. The AVD is determined 160 based on both the local and non-local signals. In some configurations the AVD is calculated based on a combination of parameters which are measured from the local and non-local cardiac signals.

Determination of the AVD in accordance with various embodiments may be implemented by either a patient-external device, such as a programmer or advanced patient management system (APM), or by an implanted cardiac device, or may be a manually implemented procedure (e.g., by using a printed table lookup to compute optimal parameters from measured conduction data). In some embodiments, the determination of the CRT pacing parameters may be accomplished using a hybrid approach wherein some processes are performed by a device and other processes are manually performed.

A wide variety of implantable cardiac monitoring and/or stimulation devices may be configured to implement determination of pacing parameters in accordance with the present invention. A non-limiting, representative list of such devices includes cardiac monitors, pacemakers, cardiovertors, defibrillators, resynchronizers, and other cardiac monitoring and therapy delivery devices. These devices may be configured with a variety of electrode arrangements, including transvenous, endocardial, and epicardial electrodes (i.e., intrathoracic electrodes), and/or subcutaneous, non-intrathoracic electrodes, including can, header, and indifferent electrodes, and subcutaneous array or lead electrodes (i.e., non-intrathoracic electrodes).

Embodiments of the present invention may be implemented in the context of a wide variety of cardiac devices, such as those listed above, and are referred to herein generally as patient-internal medical devices (PIMD) for convenience. A PIMD implemented in accordance with the present invention may incorporate one or more of the electrode types identified above and/or combinations thereof.

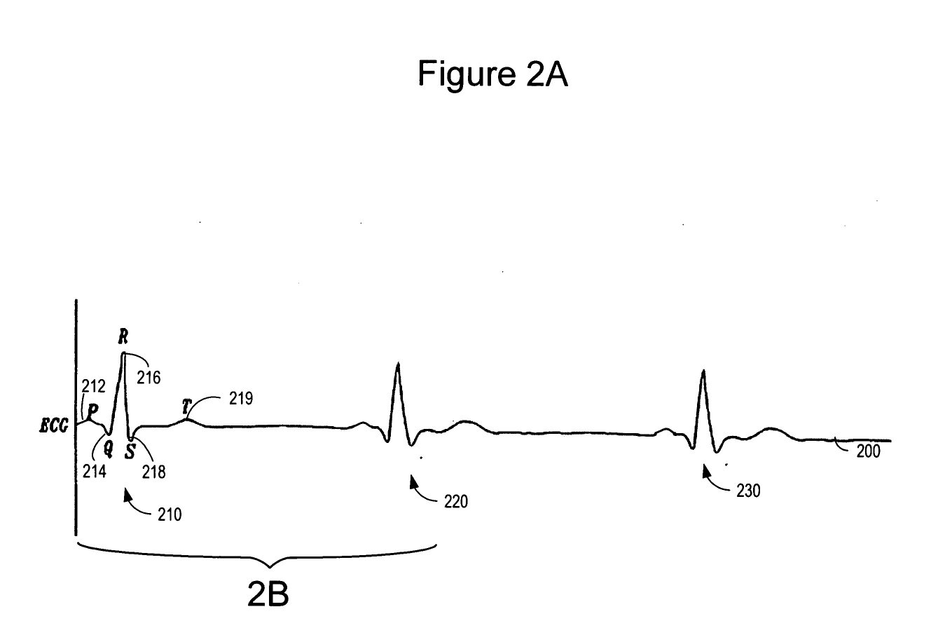

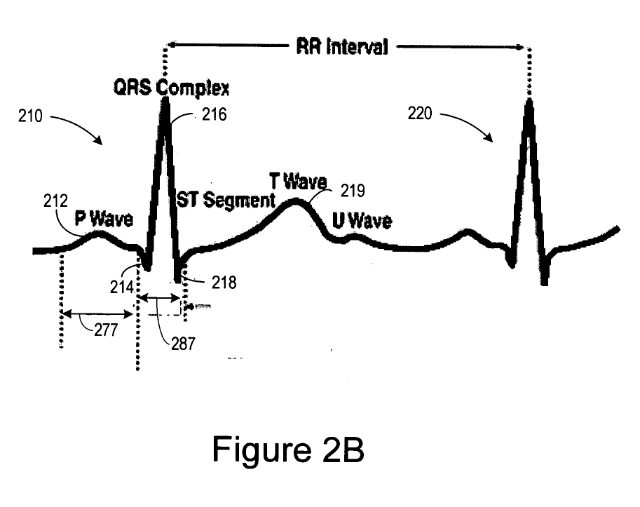

Referring to FIGS. 2A and 2B, a cardiac activation signal 200 describes the electrical activation sequence of a patient's heart as recorded, for example, by a non-local electrode pair. The graph of FIG. 2A illustrates an example of the cardiac electrical signal 200 for three heartbeats, denoted as a first heartbeat 210, a second heartbeat 220, and a third heartbeat 230. FIG. 2B is a magnified view of the first two heartbeats 210, 220 of the cardiac signal identified by bracket 2B in FIG. 2A.

Referring to the first heartbeat 210, the portion of the cardiac signal representing depolarization of the atrial muscle fibers is referred to as a P-wave 212. Depolarization of the ventricular muscle fibers is collectively represented by a Q 214, R 216, and S 218 waves of the cardiac signal 200, typically referred to as the QRS complex, which is a well-known morphologic feature of electrocardiograms. Finally, the portion of the signal representing repolarization of the ventricular muscle fibers is known as a T wave 219. Between contractions, the cardiac signal returns to an isopotential level.

The sensed cardiac signal 200 illustrated in FIGS. 2A and 2B is typical of a non-local cardiac electrical signal, effectively a superposition of all the depolarizations occurring within the heart that result in contraction. The cardiac signal 200 may be obtained directly via a non-local electrode pair. The conduction timings such as the P-wave width 277 and/or the QRS complex width 287 may be measured from the sensed cardiac signal 200. In some embodiments, the cardiac activation signal 200 used for feature measurement may be obtained indirectly, such as by interpolation between a plurality of cardiac activation signals respectively obtained from a plurality of non-local sensing electrode pairs. For example, a cardiac activation signal used for feature measurement may be obtained using a signal separation methodology.

Signal separation methodologies, such as blind source separation, are able to separate signals from individual sources that are mixed together into a composite signal. The main principle of signal separation works on the premise that spatially distributed electrodes collect components of a signal from a common origin (e.g., the heart) with the result that these components may be strongly correlated to each other. In addition, these components may also be weakly correlated to components of another origin (e.g., noise). A signal separation algorithm may be implemented to separate these components according to their sources and produce one or more cardiac activation signals based on the source separation. Methods and systems for acquiring a cardiac activation signal using blind source separation, useful in conjunction with the methods of the present invention, are described in commonly owned U.S. patent application Ser. No. 11/125,068 filed May 9, 2004 which is incorporated herein by reference.

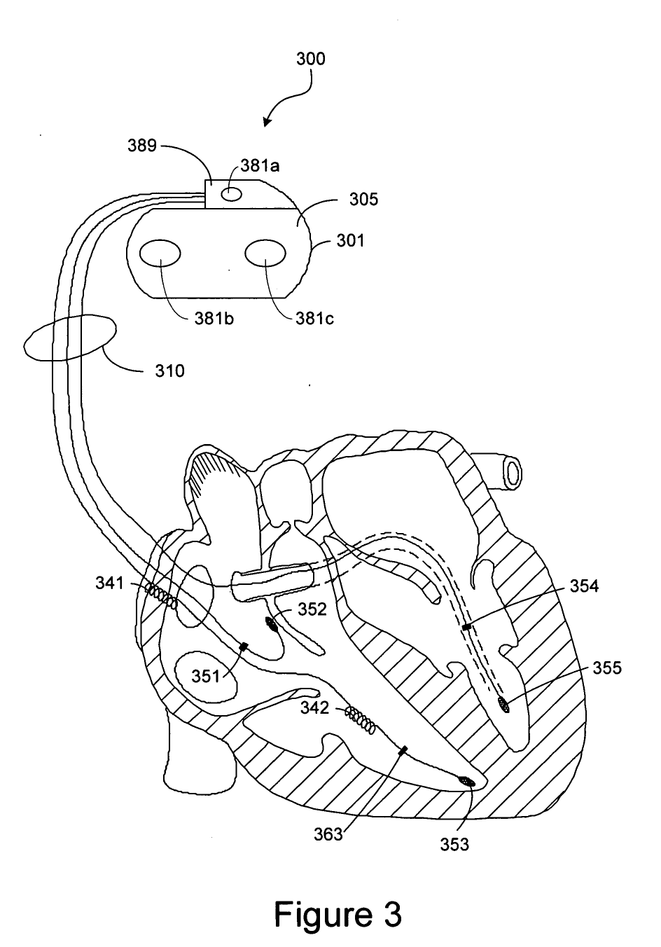

Referring now to FIG. 3, the implantable device illustrated in FIG. 3 is an embodiment of a PIMD that may be used in conjunction with AVD determination in accordance with embodiments of the invention. In this example, the implantable device includes a cardiac rhythm management device (CRM) 300 including an implantable pulse generator 305 electrically and physically coupled to an intracardiac lead system 310.

Portions of the intracardiac lead system 310 are inserted into the patient's heart. The intracardiac lead system 310 includes one or more electrodes configured to sense electrical cardiac activity of the heart and deliver electrical stimulation to the heart. Additionally, the cardiac electrodes and/or other sensors may be used to sense the patient's transthoracic impedance, and/or sense other physiological parameters, such as cardiac chamber pressure or temperature. The electrodes shown in FIG. 3 illustrate one possible arrangement of electrodes. Many other electrode arrangements, including intracardiac and/or subcutaneous intrathoracic and non-intrathoracic electrodes, may be used to achieve non-local sensing and are considered to fall within the scope of the invention. The lead system 310 may include wired and/or wirelessly coupled electrodes. In wireless configurations, sensed signals from the electrodes are wirelessly communicated to the PIMD and/or a patient-external device.

Portions of the housing 301 of the pulse generator 305 may optionally serve as one or multiple can or indifferent electrodes. The housing 301 is illustrated as incorporating a header 389 that may be configured to facilitate removable attachment between one or more leads and the housing 301. The housing 301 of the PIMD may include one or more can electrodes 381b, and 381c. The header 389 of the PIMD may include one or more indifferent electrodes 381a. The housing 301 and/or header 389 may include any number of electrodes positioned anywhere in or on the housing 301 and/or header 389. In various configurations, one or more housing/header electrodes 381a-381c may be used as one electrode of an electrode pair 381a-381c providing non-local sensing and another one or more housing/header electrodes 381a-381c may be used as thetheher electrode of the electrode pair. In other embodiments, one or more housing/header electrodes 381a-381c may be used as one electrode of an electrode pair providing non-local sensing and one or more intracardiac electrodes 341, 351, 342, 363, 354 may be used as the other electrode of the electrode pair.

Communications circuitry is disposed within the housing 301 for facilitating communication between the pulse generator 305 and a patient-external device, such as an external programmer or advanced patient management (APM) system, for example. The communications circuitry may also facilitate unidirectional or bidirectional communication with one or more implanted, external, cutaneous, or subcutaneous physiologic or non-physiologic sensors, patient-input devices and/or information systems.

The pulse generator 305 may optionally incorporate sensors that may be used to sense patient activity as well as various respiratory and cardiac related conditions. For example, sensors may be optionally configured to sense respiration, snoring, activity level, chest wall movements, rales, coughing, heart sounds, murmurs, and other information.

For example, the lead system 310 and pulse generator 305 of the CRM 900 may incorporate one or more transthoracic impedance sensors that may be used to acquire the patient's respiratory waveform, or other respiratory-related information. The transthoracic impedance sensor may include, for example, one or more intracardiac electrodes 341, 342, 351-355, 363 positioned in one or more chambers of the heart. The intracardiac electrodes 341, 342, 351-355, 363 may be coupled to impedance drive/sense circuitry positioned within the housing 301 of the pulse generator 305. Information from the transthoracic impedance sensor and/or an activity sensor may be used to adapt the rate of pacing to correspond to the patient's activity and/or hemodynamic need.

The lead system 310 may include one or more cardiac pace/sense electrodes 351-355 positioned in, on, or about one or more heart chambers for sensing electrical signals from the patient's heart and/or delivering pacing pulses to the heart. The intracardiac sense/pace electrodes 351-355, such as those illustrated in FIG. 3, may be used to sense and/or pace one or more chambers of the heart, including the left ventricle, the right ventricle, the left atrium and/or the right atrium. The lead system 310 may include one or more defibrillation electrodes 341, 342 for delivering defibrillation/cardioversion shocks to the heart.

The pulse generator 305 may include circuitry for detecting cardiac arrhythmias and/or for controlling pacing or defibrillation therapy in the form of electrical stimulation pulses or shocks delivered to the heart through the lead system 310.

In some embodiments, the pulse generator 305 may include circuitry for determining one or more pacing parameters, such as AVD, based at least in part on a cardiac activation signal sensed via a non-local electrode pair. In other embodiments, the pulse generator 305 may transfer sensed or derived information relevant to pacing parameter determination to a patient-external device. Following download of the implantably sensed or derived information, the pacing parameter determination may be made by the patient-external device or may be made by a human analyst. Following pacing parameter determination, the parameters for pacing may be used by the PIMD to control pacing pulses delivered to the heart to effect a pacing therapy for improving cardiac function.

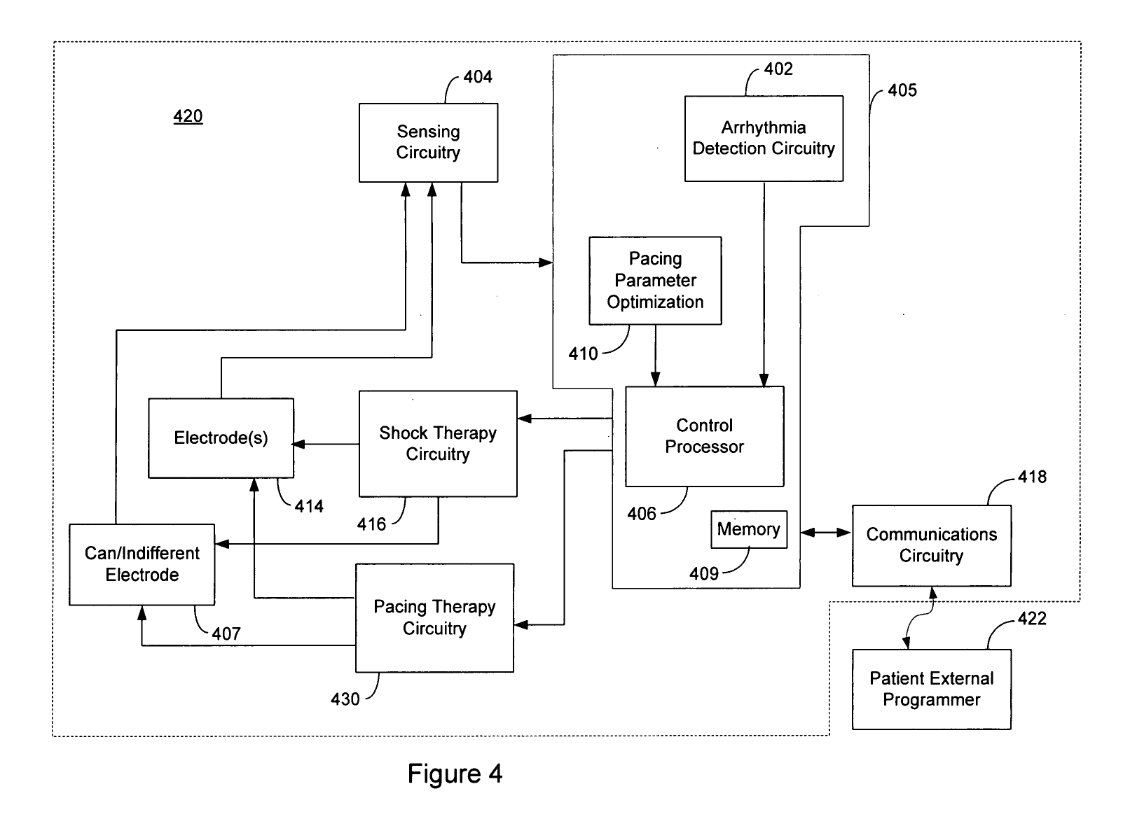

FIG. 4 is a block diagram depicting various components of a cardiac rhythm management (CRM) system incorporating a PIMD 420 and a patient-external programmer 422 in accordance with embodiments of the present invention. The components, functionality, and configurations depicted in FIG. 4 are intended to provide an understanding of various features and combinations of features that may be incorporated in such a system. It is understood that a wide variety of device configurations are contemplated, ranging from relatively sophisticated to relatively simple designs. As such, particular configurations may include some components illustrated in FIG. 4, while excluding other components illustrated in FIG. 4. In certain embodiments, the arrangement of the functional blocks may vary from the arrangement depicted in FIG. 4.

Although FIG. 4 illustrates functionality for determining AVD incorporated in the PIMD 420, in alternate embodiments, such functionality may be incorporated in the patient-external programmer 422, or may be divided between the PIMD 420 and the patient-external programmer 422. In some embodiments, the parameter optimization module may execute a relatively sophisticated algorithm that automatically determines an optimal AVD and/or other pacing parameters. In yet other embodiments, determination of the optimal pacing parameters involves formatting information for display, such as via a display, allowing a human analyst to make a determination regarding optimal pacing parameters.

Illustrated in FIG. 4 is a PIMD 420 having processor-based control system 405 which includes a control processor 406 coupled to appropriate memory (volatile and/or non-volatile) 409, it being understood that any logic-based control architecture may be used. The control system 405 is coupled to circuitry and components to sense electrical signals produced by the heart and deliver electrical stimulation energy to the heart under predetermined conditions to treat cardiac arrhythmias and/or other cardiac conditions. The electrical energy delivered by the PIMD 420 may be in the form of low energy pacing pulses or high-energy pulses for cardioversion or defibrillation.

Cardiac signals are sensed using the cardiac electrode(s) 414 and the can or indifferent electrode 407 provided on the PIMD housing. Cardiac signals may also be sensed using only the electrode(s) 414, such as in a non-active can configuration. As such, electrode sensing configurations, including non-local electrode configurations may be employed. A switch matrix (not shown) may be employed to selectably couple various combinations of the cardiac electrodes 414 and the can or indifferent electrodes 407 to the sensing circuitry 404. The sensed cardiac signals are received by sensing circuitry 404, which includes sense amplification circuitry and may also include filtering circuitry and an analog-to-digital (A/D) converter. The sensed cardiac signals processed by the sensing circuitry 404 may optionally be processed by noise reduction circuitry (not shown), which may reduce noise and or increase the signal to noise ratio (SNR) of the signals before signals are sent to other components of the PIMD 420, such as the arrhythmia detection circuitry 402, the control processor 406, and/or the pacing parameter optimization module 410. The noise reduction circuitry may operate to improve the SNR of sensed cardiac signals by removing noise content of the sensed cardiac signals introduced from various sources. Typical types of cardiac signal noise includes electrical noise and noise produced from skeletal muscles, for example. A number of methodologies for improving the SNR of sensed cardiac signals in the presence of skeletal muscular induced noise may be utilized.

Arrhythmia detection circuitry 402 may include a signal processor that coordinates analysis of the sensed cardiac signals and/or other sensor inputs to detect cardiac tachyarrhythmia. Rate based and/or morphological discrimination algorithms may be implemented by the signal processor of the detection circuitry 402 to detect and verify the presence and severity of an arrhythmic episode. The detection circuitry 402 communicates information associated with the detection of tachyarrhythmia to the control processor 406 so that the control processor 406 can coordinate delivery of an appropriate therapy, such as a defibrillation, cardioversion, or antitachyarrhythmia pacing therapy (ATP) to terminate or mitigate the arrhythmia.

A PIMD 420 may incorporate a cardiac pacing capability in addition to, or to the exclusion of, cardioversion and/or defibrillation capabilities. As is shown in FIG. 4, the PIMD 420 includes pacing therapy circuitry 430 that is coupled to the control system 405 and the electrode(s) 414 and can/indifferent electrodes 407. Upon command, the pacing therapy circuitry 430 delivers pacing pulses to the heart in accordance with a selected pacing therapy, such as a pacing therapy using the AVD determined be the approaches described herein.

Control signals, developed in accordance with a pacing regimen by pacemaker circuitry within the control system 405, are initiated and transmitted to the pacing therapy circuitry 430 where pacing pulses are generated. A pacing therapy, such as those discussed and incorporated herein, may be modified by the control system 405. In one particular application, an AVD determination approach of the present invention may be implemented to enhance CRT pacing therapy.

The sensing circuitry 404 is configured to sense at least cardiac electrical activation signals via the electrodes 414, 407 and to communicate cardiac signal information to the control system 405. The sensing circuitry 404 of the PIMD 420 shown in FIG. 4 may also be configured to receive signals from one or more additional physiologic and/or non-physiologic sensors. The additional physiological signals and/or non-physiological signals may be used in connection with various diagnostic, therapeutic or monitoring implementations. For example, the PIMD 420 may include sensors or circuitry for detecting respiratory system signals, cardiac system signals, and/or signals related to patient activity. In one embodiment, the PIMD 420 senses intrathoracic impedance, from which various respiratory parameters may be derived, including, for example, respiratory tidal volume and minute ventilation. Sensors and associated circuitry may be incorporated in connection with a PIMD 420 for detecting one or more body movement or body posture or position related signals.

The control system 405 may analyze and/or use the cardiac signals sensed via the electrodes 414, 407 for various purposes. For example, the control system 405 may initiate one or more pacing delay and/or pacing escape intervals for each cardiac cycle based on the cardiac signal information obtained from the sensing circuitry. The control system may analyze the cardiac signals to determine heart rhythm characteristics, such as the intrinsic atrial or ventricular heart rate.

The sensing circuitry 404 in conjunction with the electrodes 414, 407 obtains cardiac activation signals using local and/or non-local electrode pairs. Cardiac activation signals sensed via the local and/or non-local electrode pairs may be transferred to the pacing parameter optimization module 410 of the control system 405. In some embodiments, the pacing parameter optimization module 410 operates to estimate the conduction timing delays from the non-local cardiac activations signals, such as by measuring P-wave and/or QRS complex widths. The pacing parameter optimization module is configured to determine the AVD for pacing therapy based on the conduction timing. The AVD so determined may be used by the control system 405 to schedule pacing and may be used in conjunction with pacing therapy for cardiac resynchronization.

Memory circuitry 409 of the control system 405 contains parameters for operating in various monitoring, defibrillation, and pacing modes, and may store data indicative of cardiac signals received by the detection circuitry 402. The memory circuitry 409 may also be configured to store historical data, which may be used for various purposes and transmitted to an external receiving device as needed or desired. For example, in certain embodiments, the memory circuitry may store formulas and/or tables used in connection with pacing parameter determination. The formulas and/or tables may be indexed according to heart rate.

Communications circuitry 418 is coupled to the control processor 406 of the control system 405. The communications circuitry 418 allows the PIMD 420 to communicate with one or more patient-external devices or systems 422 situated external to the PIMD 420. In one configuration, the communications circuitry 418 and the patient-external device 422 use a wire loop antenna and a radio frequency telemetric link, as is known in the art, to receive and transmit signals and data between the patient-external device 422 and communications circuitry 418. In this manner, programming commands and data are transferred between the PIMD 420 and the patient-external device 422 during and after implant. Using a patient-external programmer, a physician is able to set or modify various parameters used by the PIMD 420. For example, a physician may set or modify parameters affecting monitoring, detection, pacing, and defibrillation functions of the PIMD 420, including pacing and cardioversion/defibrillation therapy modes.

In certain embodiments, the control processor 406 transmits information for pacing parameter determination to the patient-external device 422. The information may include, for example, cardiac electrical activation signals obtained via local and/or non-local sensing, measured characteristics or features of the signals, and/or other information. The patient-external device 422 may use the transmitted information to determine AVD and/or other pacing parameters or may format and display information related to parameter optimization to a human analyst.

Processes for determining the AVD based on cardiac activation signals obtained via non-local sensing in accordance with embodiments of the invention may be implemented in the PIMD 420, in the patient-external device 422, such as a programmer or advanced patient management system, or by a manually implementable procedure, such as by using a printed table lookup to compute the optimal values, and/or by any combination of these techniques.

For example, in one embodiment, the patient-external programmer 422 communicates with the PIMD 420 over a telemetry link and receives either raw electrogram data, markers corresponding to particular sensed events, and/or measurements of intervals between sensed events or feature widths as computed by the implantable device. The external programmer 420 may then compute optimal settings for the AVD which are either transmitted to the PIMD 420 for immediate reprogramming or presented to a clinician operating the external programmer as recommendations. Alternatively, the external programmer 422 may present the conduction data to the human analyst who then programs the PIMD 420 in accordance with an algorithm. In some implementations, the PIMD 420 is programmed to automatically set the AVD and/or other pacing parameters in accordance with information gathered from its sensing channels.

Embodiments of the present invention described herein are directed to methods for optimization of the AVD based on interatrial (RA to LA) or interventricular (RV to LV) conduction delays. The interatrial conduction delay is approximated based on the measured P-wave width of a cardiac activation signal sensed via a non-local electrode pair. The interventricular conduction delay is approximated based on the measured QRS complex width of the non-locally sensed cardiac activation signal.

Other methods of approximating conduction delays using non-local sensing are also applicable. For example, the interventricular conduction delay and/or the interatrial conduction delay may be estimated from local signals. For example, the interventricular conduction delay may be approximated by the temporal difference in the R-wave peaks of right and left ventricular electrograms, the interatrial conduction delay may be approximated by the temporal difference in the P-wave peaks of right and left atrial electrograms.

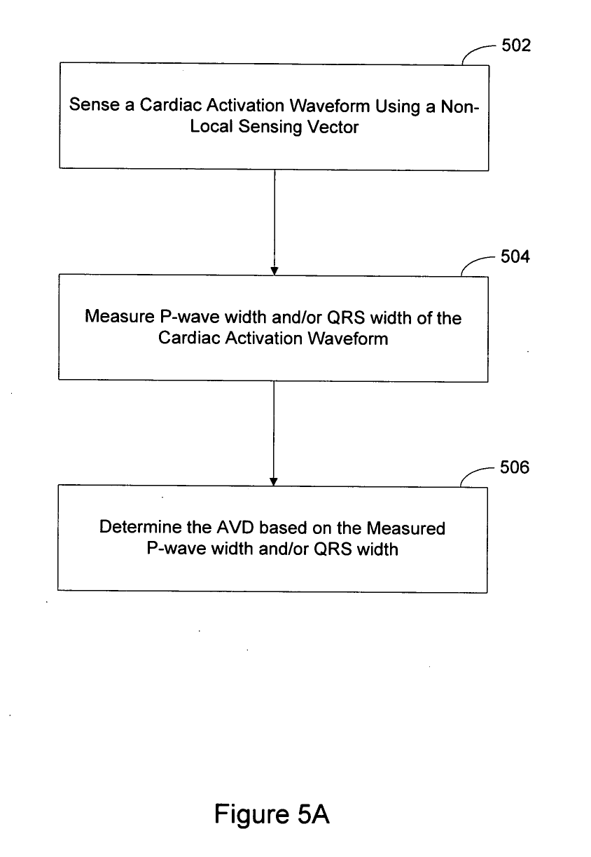

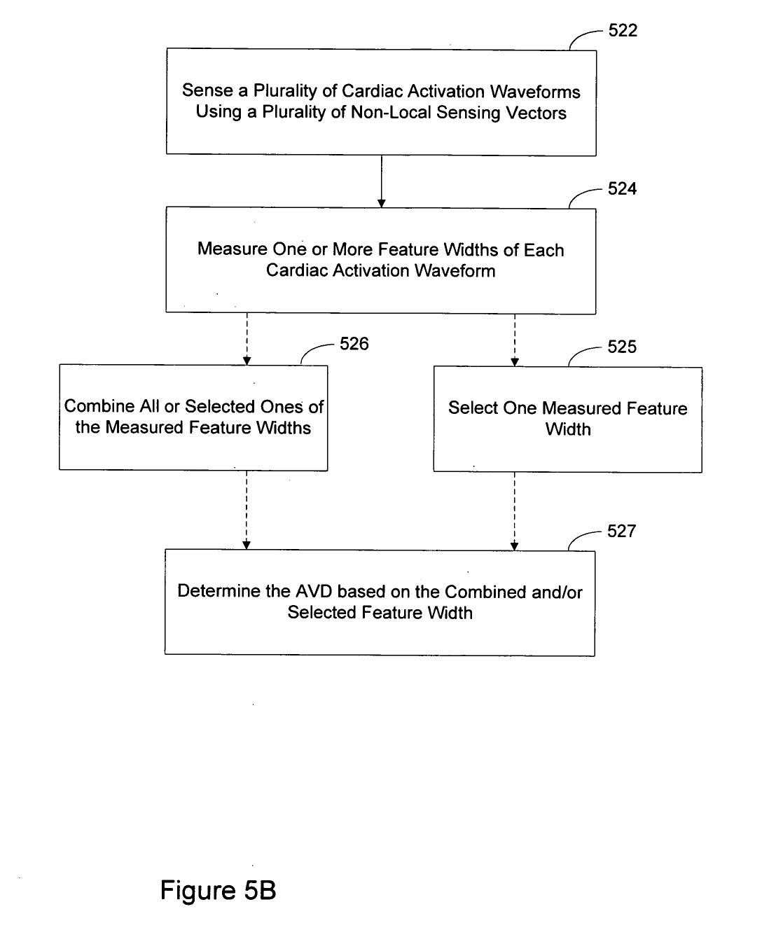

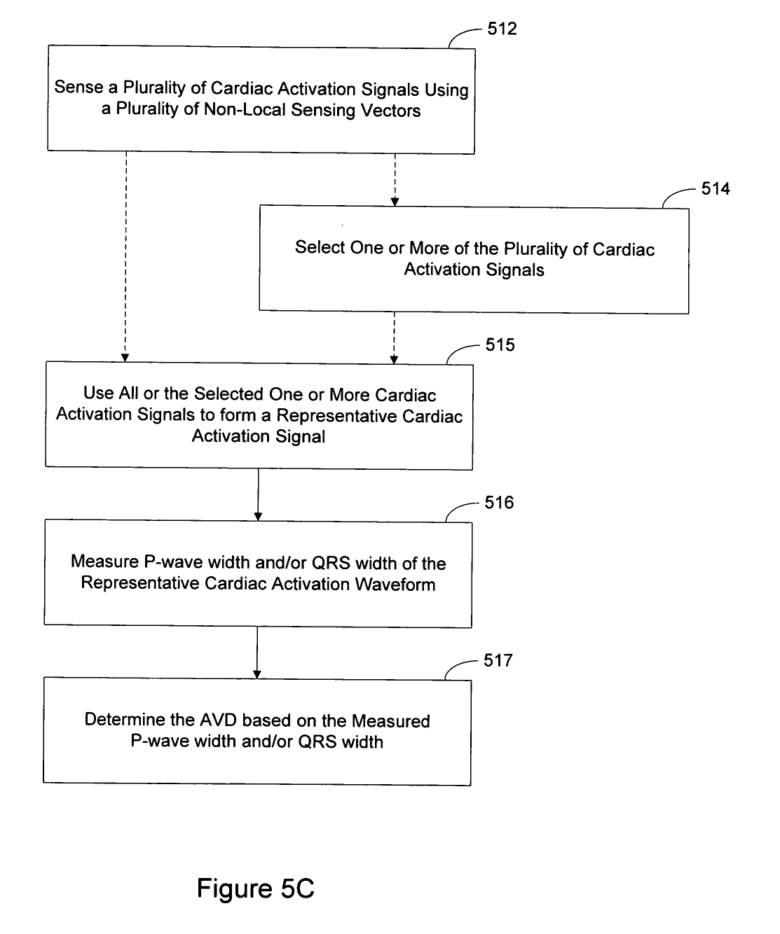

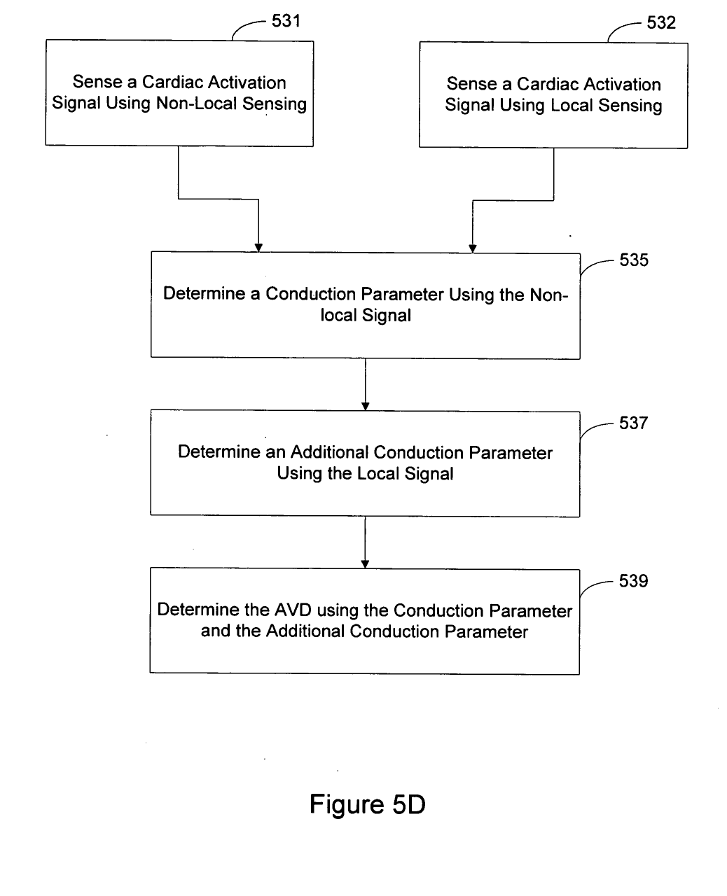

Exemplary methods in accordance with embodiments of the invention are illustrated by the flowcharts of FIGS. 5A-5D. Each of these exemplary processes uses a cardiac activation signal feature width, such as a P-wave width or QRS width to determine an AVD. As illustrated in the flowchart of FIG. 5A, a cardiac activation signal is sensed 502 via a non-local electrode pair. The widths of the P-wave and/or QRS complex of the cardiac activation signal are measured 504. The AVD is determined 506 based on the measured P-wave and/or QRS widths.

In another embodiment, illustrated in FIG. 5B, multiple cardiac electrical activation signals are sensed 522 and feature width measurement is performed 524 for each cardiac activation signal. A feature width measurement may involve measuring one or several features of the cardiac activation signal, such as the widths of the P-waves and/or QRS complexes. According to one optional process, one feature width measurement is selected 525 and used 527 for AVD determination. According to another optional process, all or selected ones of the feature width measurements are combined 526 and the combined feature widths are used 527 for AVD determination. In one example, a P-wave width of each sensed cardiac activation signal is measured and the measured P-wave widths are combined, such as by averaging. The AVD is determined based on the average P-wave width. In another example, a P-wave width and a QRS width of each sensed cardiac signal are measured. The AVD is based on an average of the measured P-wave widths and an average of the measured QRS widths. In other example, instead of combining each measured P-wave width and/or QRS width, only selected ones are combined.

In some implementations, a plurality of cardiac activation signals may be combined to form the cardiac activation signal used for feature measurement. The cardiac activation signal used for feature measurement may be a combination or interpolation of the all or certain ones of the sensed signals. As illustrated in the flowchart of FIG. 5C, a plurality of cardiac activation signals are measured 512. According to one optional process, all of the sensed cardiac activation signals are combined to form 515 a representative cardiac activation signal used for feature measurement. According to another optional process, one or more of the sensed cardiac activation signals are selected 514 and are used to form 515 the representative cardiac activation signal for feature measurement. The P-wave and/or QRS wave widths of the representative cardiac activation signal are measured 516. The AVD is determined 517 based on the measured P-wave and/or QRS widths.

Selection of cardiac signals in the processes described herein may be performed based on various factors. For example, in some embodiments the cardiac activation signals having the least noise or highest signal to noise ratio may be selected. In other embodiments, one or more cardiac activation signals identified using blind source separation may be used for feature measurement. For example, if the feature measurement involves measurement of the P-wave, the cardiac activation signal selected for feature measurement may be a measured signal or an interpolated signal associated with an optimal activation vector identified through blind source separation (BSS).

In one implementation, the cardiac activation signal used for P-wave width measurement may be acquired using one or more non-local electrode pairs that more closely align with the dominant P vector identified using BSS. The cardiac activation signal used for QRS width measurement may be acquired using one or more electrode pairs that more closely align with the dominant QRS vector identified using BSS. One of the sources separated by the BSS technique is noise, thus, the BSS technique can be used to avoid the use of noisy signals. Additionally, another BSS algorithm determines which of the separated sources is a cardiac source. The BSS technique can be used to enhance the view of the signal associated with atrial contraction (P-wave) over the QRS signal. This is particularly advantageous because in typical configurations, the P-wave is more difficult to detect than the QRS complex

In some embodiments, a conduction parameter estimated using the non-local cardiac activation signal may be used in conjunction with an additional conduction parameter determined using a local cardiac activation signal, as illustrated by the flowchart of FIG. 5D. Cardiac activation signals are sensed via a non-local electrode pair and a local electrode pair 531, 532. One or more conduction parameters, such as P-wave width and/or QRS width, are determined 535 using the non-local signal. One or more additional conduction parameters are determined 537 using the local signal. Examples of the additional conduction parameters include the intrinsic right atrioventricular interval between an atrial sense (or pace) and a right ventricular sense (AVR interval), estimation of the interatrial conduction delay by measuring the timing between P-waves detected via local RA and LA electrograms, and estimation of the interventricular conduction delay by measuring the timing between R-waves detected via local RV and LV electrograms. The AVD is determined 539 using both the conduction parameters estimated from the non-local signal or signals and the additional conduction parameters estimated from the local signal or signals.

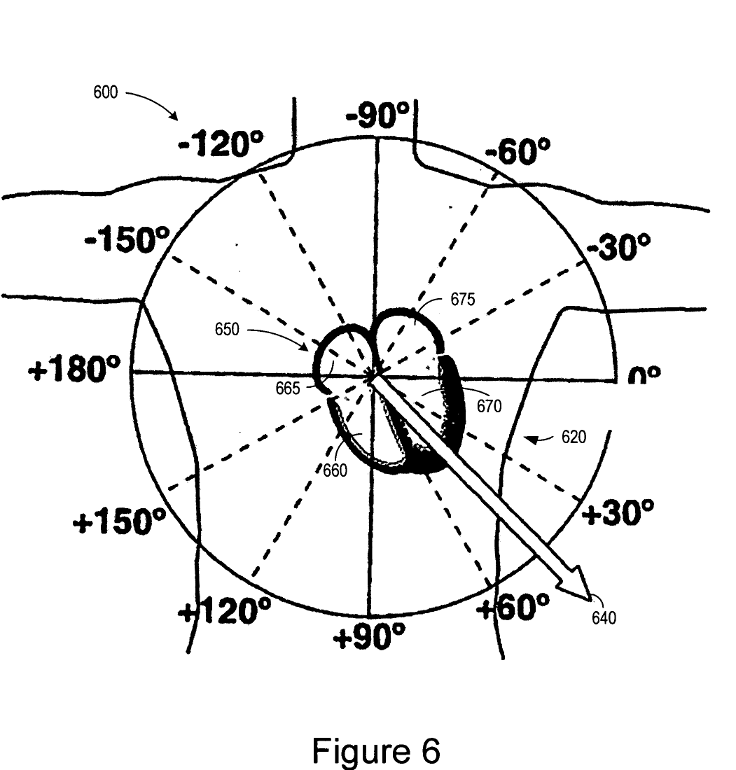

FIG. 6 illustrates a convenient reference for describing cardiac activation vectors associated with a depolarization wavefront which may be sensed via local or non-local electrode pairs. FIG. 6 is a polar plot 600 of a cardiac activation vector 640 superimposed over a frontal view of a thorax 620, with the origin of the polar plot located at a patient's heart 650, specifically, the atrioventricular (AV) node of the heart 650. Two major pumps operate in the heart, and they are a right ventricle 660, which pumps blood into pulmonary circulation, and a left ventricle 670, which pumps blood into the systemic circulation. Each of these pumps is connected to its associated atrium, the right atrium 665 and a left atrium 675.

The cardiac vector 640 is describable as having an angle, in degrees, about a circle of the polar plot 600, and having a magnitude, illustrated as a distance from the origin of the tip of the cardiac vector 640. The polar plot 600 is divided into halves by a horizontal line indicating 0 degrees on the patient's left, and +/−180 degrees on the patient's right, and further divided into quadrants by a vertical line indicated by −90 degrees at the patient's head and +90 degrees on the bottom. The cardiac vector 340 is projectable onto the two-dimensional plane designated by the polar plot 300.

The cardiac vector 640 is a measure of all or a portion of the projection of a heart's activation sequence onto the polar plot 600. The heart possesses a specialized conduction system that ensures, under normal conditions, that the overall timing of ventricular and atrial pumping is optimal for producing cardiac output, the amount of blood pumped by the heart per minute. The normal pacemaker of the heart is a self-firing unit located in the right atrium called the sinoatrial node. The electrical depolarization generated by this structure activates contraction of the two atria. The depolarization wavefront then reaches the specialized conduction system using conducting pathways within and between the atria. The depolarization is conducted to the atrioventricular node, and transmitted down a rapid conduction system composed of the right and left bundle branches, to stimulate contraction of the two ventricles.

The normal pacemaker and rapid conduction system are influenced by intrinsic automatic activity and by the autonomic nervous system, which modulates heart rate and the speed with which electrical depolarizations are conducted through the specialized conduction system. There are many diseases that interfere with the specialized conduction system of the heart, and many result in abnormally fast, slow, or irregular heart rhythms.

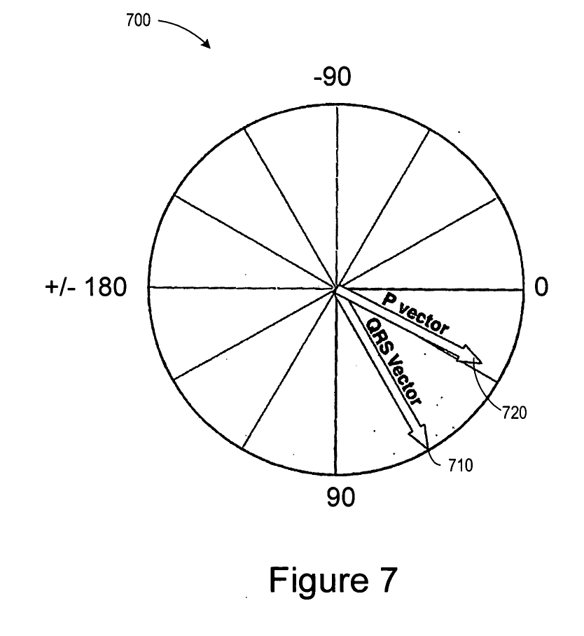

The cardiac activation vector 640 may be, for example, associated with the entire cardiac cycle, and describe the mean magnitude and mean angle of the cardiac cycle. Referring now to FIG. 7, a polar plot 700 is illustrated of separate portions of the cardiac cycle that may make up the cardiac activation vector 640 of FIG. 6. As is illustrated in FIG. 7, a QRS vector 710 and a P vector 720 are illustrated having approximately 60 degree and 30 degree angles, respectively. The QRS vector 710 represents the projection of the mean magnitude and angle of the depolarization wavefront during the QRS portion of the cardiac cycle onto the polar plot 400. The P vector 720 represents the projection of the mean magnitude and angle of the depolarization wavefront during the P portion of the cardiac cycle onto the polar plot 700. The projection of any portion of the depolarization wavefront may be represented as a vector on the polar plot 700.

Further, any number of cardiac cycles may be combined to provide a statistical sample that may be represented by a vector as a projection onto the polar plot 700. Likewise, portions of the cardiac cycle over multiple cardiac cycles may also be combined, such as combining a weighted summation of only the P portion of the cardiac cycle over multiple cardiac cycles, for example.

As described above, cardiac activation vectors obtained from selected portions of an electrocardiogram may be determined. In general, it may be desirable to define one or more detection windows associated with particular segments of a given patient's cardiac cycle. The detection windows may be associated with cardiac signal features, such as P wave and/or QRS complex features, for example. The detection windows may also be associated with other portions of the cardiac cycle that change in character as a result of changes in the pathology of a patient's heart. Such detection windows may be defined as fixed or triggerable windows. Further discussion of the use of BSS techniques for determining cardiac activation vectors is described in previously incorporated U.S. patent application Ser. No. 11/125,068.

In some embodiments, optimal values of the AVD may be determined as a linear or non-linear function of the conduction delays estimated using cardiac activation signals obtained via non-local sensing. In certain implementations of the techniques described herein, separate AVDs may determined for the AVD used following an atrial sense and the AVD used following an atrial pace. It will be appreciated that when both ventricles are paced, atrioventricular delays from the first atrial sense or pace to the ventricular pace for both left (AV_{L}) and right (AV_{R}) ventricles may be specified. Methods and systems for determination of optimal values for AVD and/or other pacing parameters are described in commonly owned U.S. Patent Publication 2005/0137632 which is incorporated herein by reference.

In various aspects of the present invention, an optimum AVD interval may be computed using a linear function of the conduction delay between right atrial and left atrial activation and the conduction delay between right and left ventricular activation. In this embodiment, the atrial conduction delay is approximated using the P-wave width (Pwidth) of a cardiac activation signal measured via a non-local electrode pair. The ventricular conduction delay is approximated using the QRS complex width of the non-local signal. An optimal AVD for a particular patient may be estimated from the approximated conduction delays in terms of specified coefficients Kₙ as:

AVD=K₁(Pwidth)+K₂AV_{L}+K₃AV_{R}+K₄

or

AVD=K₅(Pwidth)+K₆AV_{R}+K₇(Δ_{RL})+K₈

or

AVD=K₉(AP)+K₁₀AV_{L}+K₁₁AV_{R}+K₁₂

or

AVD=K₁₃(AP)+K₁₄AV_{R}+K₁₅(Δ_{RL})+K₁₆

where Pwidth is the P-wave width of a cardiac activation signal which can be measured via a non-local electrode pair, Δ_{RL} is the width of the QRS complex which can be measured via the non-local pair, and A_{VR} is the right intrinsic atrio-ventricular interval between an atrial sense (or pace) and a right ventricular sense which can be measured via a local electrode pair. AV_{L} is the right intrinsic atrio-ventricular interval between an atrial sense (or pace) and a left ventricular sense which can be measured via a local electrode pair.

Derivation of the specified coefficients, Kₙ, for later programming into the system or for use by a clinician, involves obtaining clinical population data related to particular values of the measured conduction parameters to an optimum value of the pacing parameter as determined by concurrent measurement of another parameter reflective of cardiac function (e.g., maximum dP/dt). A linear regression analysis may be performed to derive values of the specified coefficients used in the formula for setting the pacing parameter, the specified coefficients thus being regression coefficients.

According to another implementation of the present invention, the optimal AVD may be computed as a function of the interval from the P-wave detected via a local right atrial electrogram and the end of the non-local P-wave (AP). This implementation is particularly useful for enhancing stroke volume. The AVD for this embodiment may be computed as follows:

AVD=K₁₇(AP)+K₁₈

or

AVD=K₁₉(Pwidth)+K₂₀

The coefficients of the equation may be obtained as before from a regression analysis of clinical population data to achieve enhanced cardiac function. The techniques above may be implemented using cardiac activation signals generated from signals sensed via one or more non-local electrode pairs and/or one or more local electrode pairs. After obtaining an AVD as described above, the BVO may be set to either a nominal value or computed from conduction data as is known in the art.

As previously discussed, the techniques for determining AVD values as described above, as well as others to be described below, may be implemented in a number of different ways. In one implementation, a system for setting the pacing parameters includes an external programmer. In an example embodiment, one or more intrinsic conduction parameters, as measured from electrogram signals generated by the sensing channels of an implantable cardiac resynchronization device during intrinsic beats, are transmitted to the external programmer via a wireless telemetry link. The measured conduction data, such as P-wave width and/or QRS width, may represent averages of values obtained during a specified number of beats. The external programmer then computes the AVD in accordance with a formula that equates an optimum value of the AVD to a linear sum of the measured conduction parameters multiplied by specified coefficients. In an automated system, the external programmer then automatically programs the implantable device with the computed AVD, while in a semi-automated system the external programmer presents the computed AVD to a clinician in the form of a recommendation.

In another automated application, an automated system may also be made up of the implantable device alone which collects conduction data, computes the AVD, and then sets the parameters accordingly.

In another embodiment, which may be referred to as a manual system, the external programmer presents the collected intrinsic conduction data to a clinician who then programs the implantable device with parameters computed from the intrinsic conduction data by, for example, using a printed lookup table and procedure. Unless otherwise specified, references to a system for computing or setting pacing parameters throughout this document should be taken to include any of the automated, semi-automated, or manual systems just described.

Determination of AVD by the processes discussed above provide for computation of the AVD without regard to the atrial rate. For optimum hemodynamics, however, the AVD should vary with atrial rate in a manner similar to the way the intrinsic AV interval varies with atrial rate in normal individuals. Since improvement in hemodynamics is the objective of CRT, it would be desirable for a programmed pacing delays used in delivering CRT to mimic the physiological varying of the intrinsic AV interval. Varying the AVD is especially useful during high atrial rates imposed by rate-adaptive pacing modes at high exertion levels or atrial overdrive pacing algorithms.

In accordance with one aspect, the AVD may be varied according to rate by programming the implantable device with a look-up table or function which maps particular atrial rates to particular optimal AVD values. As the atrial rate changes, either due to atrial pacing or intrinsic atrial activity, the device may dynamically compute optimal AVD values for the atrial rate. If a table is used, for atrial rates which fall between values stored in the table, a linear or non-linear interpolating function may be used. Such a table or function may be generated in various ways. One way is during clinical hemodynamic testing where the AVD intervals which result in a maximized cardiac function parameter are determined for different atrial rates. Examples of cardiac function parameters which could be used in this procedure include dP/dt, arterial pulse pressure (e.g., finger pulse pressure), and measurements of cardiac output. A system made up of an external programmer and an implantable device or the implantable device alone may use intrinsic conduction data collected by the implantable device's sensing channels to compute optimal AVD for a plurality of atrial rates.

In an exemplary embodiment, the system computes optimal AVD values to be used for delivering pacing therapy to a particular patient by collecting conduction data at different atrial rates. The atrial rate may vary intrinsically or as a result of pacing during the data collection. Such conduction data may include, for example, an intrinsic AV interval measured as the interval from an atrial sense or pace to a ventricular sense, the atrial conduction delay as approximated by the P-width of a cardiac activation signal sensed using a non-local electrode, and/or the ventricular conduction delay as approximated by the width of the QRS complex of a cardiac activation signal sensed using a non-local electrode.

The system computes an optimal value of the AVD for delivering resynchronization therapy, such as by computing a linear function of the measured intrinsic AV interval, the P-wave width, and/or QRS width in the same manner as described earlier. The computed optimal AVD, along with other pacing parameters are associated with an atrial rate corresponding to the measured atrial rate which was present when the conduction data was measured, with the parameter values and their associated atrial rate being stored in a table contained in the memory of the implantable device. After generating a table in this manner (e.g., after an AVD and optionally other pacing parameters have been determined for at least two atrial rates, the implantable device may vary the pacing parameters as the atrial rate changes by determining a present atrial rate and setting the pacing parameter(s) to the optimum pacing parameter values stored in the table which are associated with the present atrial rate. In one variation of the embodiment, the atrial rate associated with each AV value stored in the table is a range defined by upper and lower limits. The implantable device then varies the AVD with the atrial rate by selecting an optimum value from the table which is associated with a range which encompasses the present atrial rate. In another variation, the implantable device determines the AVD if there is no associated atrial rate in the table corresponding to the present atrial rate by interpolating between two values stored in the table which have associated atrial rates above and below the present atrial rate using a linear or non-linear interpolation function. In still another variation, the implantable device is programmed to update the table if there is no associated atrial rate in the table corresponding to the present atrial rate by measuring conduction data, computing an optimal value for the AVD as a linear function of the P-wave width, intrinsic AV interval and/or QRS complex width, and storing the computed optimal value or values and an associated atrial rate corresponding to the present atrial rate in the table.

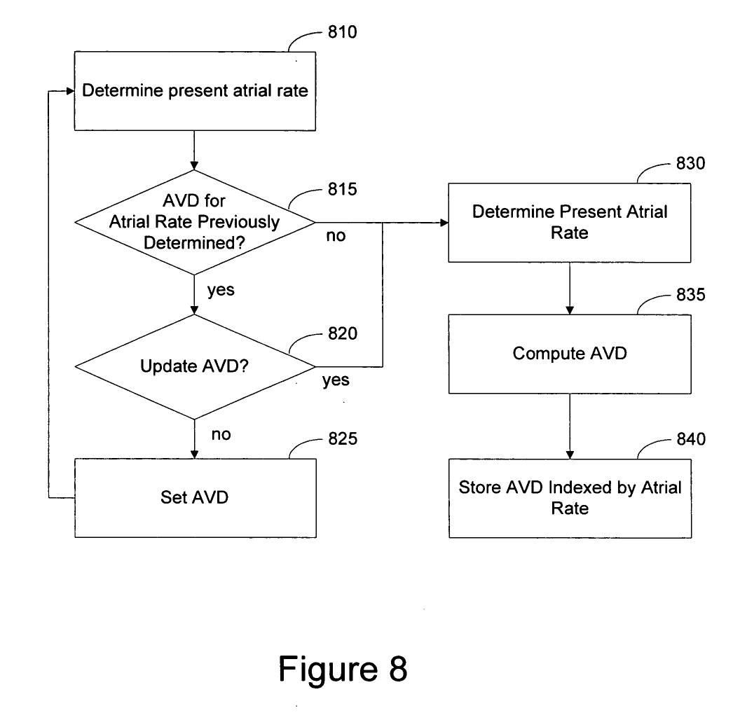

FIG. 8 illustrates an exemplary algorithm which may be implemented for setting an AVD of an implantable cardiac rhythm management device. The present atrial rate is determined 810. If a value for the AVD was previously stored 815 for the atrial rate, and an update of the AVD is not desired 820, then the AVD is set 825 to the stored value. In some implementations, if there is no associated atrial rate in the table corresponding to the present atrial rate, the AVD may be set by interpolating between two AVD values stored in the table which have associated atrial rates above and below the present atrial rate.

If the AVD for the present atrial rate was not previously determined 810, or if an update is desired 820, then conduction data is acquired 830. For example, the conduction data acquired may involve acquiring local data, such as the intrinsic AV interval measured via the local electrode pair, and non-local data such as the P-wave width and/or the QRS width as measured via the non-local electrode pair. If the intrinsic AV interval is acquired, a temporary cessation of pacing therapy is required so that intrinsically conducted ventricular beats can occur. An optimal value for the AVD is computed 835, such as by computing a linear function based on the acquired data. The computed optimal AVD value and an associated atrial rate corresponding to the present atrial rate are stored 840 in the table and the AVD is set 825 to the computed value.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. A method for determining an atrioventricular delay (AVD) for cardiac pacing therapy, comprising:
sensing, using body-implantable electrodes, at least one cardiac activation signal using a cardiac electrode pair providing non-local sensing;
estimating a conduction delay using the at least one non-local cardiac activation signal; and
determining the AVD based on the conduction delay.

2. The method of claim 1, wherein estimating the conduction delay comprises measuring a P-wave width.

3. The method of claim 1, wherein estimating the conduction delay comprises measuring a QRS complex width.

4. The method of claim 1, wherein:
sensing the at least one cardiac activation signal comprises sensing a plurality of cardiac activation signals using a plurality electrode pairs, each electrode pair providing non-local sensing; and
estimating the conduction delay comprises:
selecting a cardiac activation signal of the plurality of cardiac activation signals; and
measuring a feature of the selected cardiac activation signal.

5. The method of claim 1, wherein:
sensing the at least one cardiac activation signal comprises sensing a plurality of cardiac activation signals using a plurality electrode pairs, each electrode pair providing non-local sensing; and
estimating the conduction delay comprises:
selecting at least two cardiac activation signals of the plurality of cardiac activation signals;
combining the at least two cardiac activation signals to form a combined cardiac activation signal; and
measuring a signal feature of the combined cardiac activation signal.

6. The method of claim 1, wherein determining the AVD comprises determining the AVD as a function of the conduction delay.

7. The method of claim 1, wherein determining the AVD comprises determining the AVD as a linear combination of factors including the conduction delay.

8. The method of claim 1, further comprising:
determining an atrial rate associated with the AVD; and
storing the AVD indexed by the atrial rate.

9. The method of claim 1, further comprising delivering cardiac resynchronization therapy using the AVD.

10. The method of claim 1, wherein determining the AVD is performed at least in part by a human analyst.

11. The method of claim 1, wherein determining the AVD is performed at least in part by a processor.

12. The method of claim 1, further comprising:
sensing, using body-implantable electrodes, at least one cardiac activation signal using a cardiac electrode pair providing local sensing; and
determining an additional conduction parameter based on the local signal, wherein determining the AVD comprises determining the AVD using the conduction delay and the additional conduction parameter.

13. A cardiac rhythm management system, comprising:
at least one pair of implantable electrodes configured to electrically couple to a heart and to provide non-local sensing of cardiac electrical activity;
sense circuitry configured to sense at least one cardiac activation signal from the at least one electrode pair; and
a processor configured to estimate a conduction delay based on the at least one cardiac activation signal and to determine an AVD for cardiac pacing therapy based on the conduction delay.

14. The system of claim 13, wherein the pacing therapy comprises biventricular pacing therapy.

15. The system of claim 13, wherein at least one electrode of the electrode pair comprises an intracardiac electrode.

16. The system of claim 13, wherein at least one electrode of the electrode pair comprises a subcutaneous, non-intrathoracic electrode.

17. The system of claim 13, wherein the processor is configured to determine the conduction delay based on a measured feature of the at least one cardiac activation signal.

18. The system of claim 13, wherein the processor is configured to determine a plurality of AVDs, each AVD associated with an atrial rate.

19. The system of claim 13, wherein the processor is patient-external.

20. The system of claim 13, wherein the processor is implantable.

21. The system of claim 13, further comprising one or more additional electrodes, the one or more additional electrodes configured to electrically couple to a heart and to provide local sensing of cardiac electrical activity, wherein the sense circuitry is further configured to sense at least one local cardiac activation signal via the one or more additional electrodes and the processor is configured to estimate an additional conduction parameter based on the local cardiac activation signal and to determine the AVD based on the conduction delay and the additional conduction parameter.

22. A cardiac rhythm management system, comprising:
at least one pair of implantable electrodes configured to electrically couple to a heart and to provide non-local sensing of cardiac electrical activity;
sense circuitry configured to sense at least one cardiac activation signal from the at least one electrode pair;
means for estimating a conduction delay based on the at least one cardiac activation signal; and
means for determining at least one pacing parameter for cardiac pacing therapy based on the conduction delay.

23. The system of claim 22, further comprising:
means for sensing, using body-implantable electrodes, at least one cardiac activation signal using a cardiac electrode pair providing local sensing; and
means for estimating an additional conduction parameter based on the local signal, wherein determining the pacing parameter comprises determining the pacing parameter using the conduction delay and the additional conduction parameter.

24. The system of claim 22, further comprising:
means for determining an atrial rate associated with the AVD; and
means for storing the AVD indexed by the atrial rate.
